(19) United States
(12) Patent Application Publication
(10) Pub. No.: US 2025/0172557 A1
(43) Pub. Date: May 29, 2025

(54) METHODS FOR IDENTIFYING LILRB LIGANDS AND BLOCKING ANTIBODIES

(71) Applicant: THE BOARD OF REGENTS OF THE UNIVERSITY OF TEXAS SYSTEM, Austin, TX (US)

(72) Inventors: Chengcheng ZHANG, Dallas, TX (US); Zhiqiang AN, Houston, TX (US); Ningyan ZHANG, Houston, TX (US); Yubo HE, Dallas, TX (US); Ryan HUANG, Dallas, TX (US); Jaehyup KIM, Dallas, TX (US)

(73) Assignee: THE BOARD OF REGENTS OF THE UNIVERSITY OF TEXAS SYSTEM, Austin, TX (US)

(21) Appl. No.: 18/842,528

(22) PCT Filed: Mar. 1, 2023
(86) PCT No.: PCT/US2023/063442

§ 371 Date: Aug. 29, 2024

Related U.S. Application Data
(60) Provisional application No. 63/315,187, filed on Mar. 1, 2022.

(51) Int. Cl. G01N 33/566; C12Q 1/6897; G01N 33/58

(52) U.S. Cl. CPC G01N 33/566; C12Q 1/6897; G01N 33/582; G01N 2333/70503; G01N 2500/10

(57) ABSTRACT

Disclosed are the identification of multiple proteins that can bind to LILR receptors. based on which a screening system for LILR antagonist and blocking antibodies has been developed.

### PRIORITY CLAIM

This application claims benefit of priority to U.S. Provisional Application Ser. No. 63/315,187, filed Mar. 1, 2022, the entire contents of which are hereby incorporated by reference.

### STATEMENT REGARDING FEDERALLY FUNDED RESEARCH

The invention was made with government support under Grant No. CA248736 and CA263079 awarded by the National Institutes of Health. The government has certain rights in the invention.

### BACKGROUND

### I. Field

The present disclosure relates generally to the field of molecular biology. More particularly, it concerns methods and compositions for identifying LILRB antibodies.

### II. Description of Related Art

Immunotherapy holds great promise to achieve long-lasting anti-tumor effects. Immune checkpoint PD-1 and CTLA-4 blockade therapies have been successful in treating some types of cancers but not others. These immunotherapies target inhibitory molecules on T cells to reactivate dysfunctional T cells within the tumor microenvironment (TME). Other populations of immune cells, including myeloid cells, are present in the TME in even larger numbers than T cells. While these innate cells possess the capacity to kill tumor cells and to prime or reactivate T cells, they become dysfunctional in TME and turn into immunosuppressive myeloid cells, including monocytic myeloid-derived suppressor cells (M-MDSCs), polymorphonuclear MDSCs (PMN-MDSCs), tumor-associated macrophages (TAMs), and certain groups of tolerogenic dendritic cells, neutrophils, and eosinophils. Given the plasticity of myeloid cells, reprogramming these immune-suppressive myeloid cells into pro-inflammatory is becoming an attractive anti-cancer therapeutic strategy. New molecular targets and effective strategies are urgently needed.

Leukocyte immunoglobulin-like receptors (LILRs) represent a group of immunoregulatory proteins that are composed of five activating receptors (LILRA1, LILRA2, LILRA4, LILRA5, and LILRA6 that contain immunoreceptor tyrosine-based activation motif (ITAMs)), five inhibitory receptors (LILRB1, LILRB2, LILRB3, LILRB4, and LILRB5 that contain immunoreceptor tyrosine-based inhibitory motifs (ITIMs)), and one soluble protein (LILRA3). Recently, it has been shown that some LILR members including LILRBs and a related ITIM-containing receptor, LAIR1, have tumor-promoting functions in various hematopoietic and solid cancers. ITIM-containing receptors are expressed on a wide range of immune cells and transduce signals by recruitment of phosphatases SHP-1, SHP-2, or SHIP, leading to negative regulation of immune cells. Similar to the CTLA4 and PD-1 pathways, the LILRB pathways are considered to function as immune checkpoints. LILRBs may inhibit activities of a number of immune cell types facilitating tumor immune escape.

LILR members are primate and human specific, while there are two mouse relatives: paired immunoglobulin-like receptor A (PirA), paired immunoglobulin-like receptor B (PirB), and gp49B1. The related ITIM-containing receptor, LAIR1, has both human and mouse members. Because of the limited value of mouse models and the fact that ligands for several

LILRs are unknown, the biological function and clinical significance of these receptors remain poorly understood.

### SUMMARY

Embodiments of the present disclosure provide methods and compositions concerning modulation of LILRs activation through respective ligand(s). In a first embodiment, there is provided a method of identifying a modulator of LILR activation comprising: (a) contacting a reporter cell with a ligand of LILR and a candidate substance with potential to modulate LILR activation; and (b) detecting a level of LILR activation in the reporter cell, wherein a change in LILR reporter activation as compared to a reference level indicates that the candidate substance is a modulator of LILR activation. In certain aspects, the reporter cell is a mouse T-cell hybridoma cell.

In some aspects, the reporter cell expresses a receptor comprising the extracellular domain of LILR. In certain aspects, the extracellular domain of LILRA is further defined as the extracellular domain of LILRA1, LILRA2, LILRA3, LILRA4, and LILRA5. In particular aspects, the LILRA is further defined as LILRA1, LILRA3, and LILRA6. In particular aspects, the ligand of LILRA1 is Galectin-1, galectin-3, galectin-4, galectin-7, galectin-8, galectin-10, galectin-12, or galectin-13. In particular aspects, the ligand of LILRA3 is galectin-1, galectin-4, galectin-7, galectin-8, galectin-12, or galectin-13. In particular aspects, the ligand of LILRA6 is galectin-4 or galectin-7. In certain aspects, the extracellular domain of LILRB is further defined as the extracellular domain of LILRB1, LILRB2, LILRB3, LILRB4, LILRB5, LAIR1 (human or mouse), PirB, or gp49B1. In particular aspects, the LILRB is further defined as LILRB1, LILRB2, LILRB3, LILRB4, and LILRB5. In particular aspects, the ligand of LILRB1 is galectin-4 or galectin-7. In particular aspects, the ligand of LILRB2 is Cystatin SA (CST2), EPH Receptor A4 (EphA4), EPH Receptor A7 (EphA7), Galectin-1, claudin 22 (CLDN22), Galectin-1, galectin-2, galectin-3, galectin-4, galectin-5, galectin-7, galectin-8, galectin-12, or galectin-13. In particular aspects, the ligand of LILRB3 is galectin-4 or galectin-7. In particular aspects, the ligand of LILRB4 is SCG2, galectin-4, galectin-5, or galectin-7. In particular aspects, the ligand of LILRB5 is EphA4, EphA7, CST2, CLDN22, elastin, galectin-1, galectin-2, galectin-3, galectin-4, galectin-5, galectin-7, galectin-8, or galectin-12. In additional aspects, the receptor is a chimeric receptor comprising the intracellular domain of paired immunoglobulin-like receptor β (PILRβ).

In certain aspects, the chimeric receptor is expressed in the reporter cell through a viral expression vector. In some aspects, the viral expression vector is a retroviral expression vector. In particular aspects, the level of LILR activation is detected based on the morphology or mobility of the cell. In certain aspect, the reporter cell further comprises a reporter gene that encodes a protein that emits or generates a detectable label and is operably linked to a promoter regulated by activation of the receptor. In specific aspects, the promoter is a nuclear factor of activated T cells (NFAT) promoter. In specific aspects, the promoter is a CCL2 promoter, a CCL4 promoter, a CCLS promoter, an IL-6R promoter, an IL-8 promoter, a gp130 promoter, a OSM promoter, a TIMP-½ promoter, a TNF-R1/II promoter, a uPAR promoter or an arginase-1 promoter.

In some aspects, the detectable label is a colorometric label, fluorescent label, bioluminescent label, or chemiluminescent label. In certain aspects, the protein encoded by the report gene is GFP, YFP, RFP or Luciferase. In some aspects, the detectable label is D-luciferin. In particular aspects, the protein encoded by the reporter gene is GFP. In some aspects, the detecting step comprises flow cytometry analysis or quantification of luminescence.

In certain aspects, the candidate compound is an antibody. In some aspects, the antibody is a monoclonal antibody, a chimeric antibody, a CDR-grafted antibody, a humanized antibody, a Fab, a Fab′, a F(ab′)2, a Fv, or a scFv. In particular aspects, the antibody is a monoclonal antibody.

In certain aspects, an increase in the level of LILR reporter expression as compared to the reference level indicates that the modulator is an agonist. In certain aspects, a decrease in the level of LILR reporter expression as compared to the reference level indicates that the modulator is an antagonist. In certain aspects, the candidate substance is linked to a substrate. In certain aspects, the candidate substance is linked to a cell expressing FcR.

A further embodiment provides a composition for identifying a modulator of LILR activation. In one aspect, the composition comprises a candidate LILR modulator, the ligand of LILR and a reporter cell expresses a receptor comprising an extracellular domain of LILR, wherein the reporter cell has a phenotype indicating LILR activation. In certain aspects, the reporter cell further comprises a reporter gene that encodes a protein that emits or generates a detectable label and is operably linked to a promoter regulated by activation of the receptor. In some aspects, the receptor further comprises an intracellular domain of PILRβ. In certain aspects, the candidate LILR inhibitor is an antibody. In some aspects, the protein encoded by the reporter gene is GFP. In certain aspects, the composition further comprises a cell expressing FcR.

A further embodiment provides a composition for identifying a modulator of LILR activation in the absence of its known ligands. In one aspect, the composition comprises a candidate LILR modulator, and a reporter cell expresses a receptor comprising an extracellular domain of LILR, wherein the reporter cell has a phenotype indicating LILR activation. In certain aspects, the reporter cell further comprises a reporter gene that encodes a protein that emits or generates a detectable label and is operably linked to a promoter regulated by activation of the receptor. In some aspects, the receptor further comprises an intracellular domain of PILRβ. In certain aspects, the candidate LILR inhibitor is an antibody. In some aspects, the protein encoded by the reporter gene is GFP. In certain aspects, the composition further comprises a cell expressing FcR.

An even further embodiment provides a method of treating cancer in a subject comprising administering an effective amount of an inhibitor of LILR activation (e.g., identified by the embodiments disclosed herein) to a subject. In some aspects, the inhibitor of LILR activation is an antibody. In particular aspects, the cancer is AML.

A further embodiment provides a method of treating autoimmune disease or inhibiting the onset of transplant rejection or treating an inflammatory disorder in a subject comprising administering an effective amount of an agonist of ligand-induced LILR reporter activation to a subject.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1: CST2 activates LILRB2 chimeric receptor reporter cells. Proteins (SEMA4A as a positive control, and CST2) were coated at 20 μg/mL for 18 hours at 37° C. Protein coated wells were washed with PBS three times to remove unbound proteins. Soluble IgG or an anti-LILRB2 blocking antibody (Anti-RB2) was added at 20 μg/mL along with 50,000 LILRB2 chimeric receptor reporter cells (B2RC) and incubated for 24 hours. GFP⁺ cells were measured by flow cytometry.

FIG. 2: EphA4 activates LILRB2 chimeric receptor reporter cells. Proteins (SEMA4A as a positive control, and EphA4) were coated at 20 μg/mL for 18 hours at 37° C. Protein coated wells were washed with PBS three times to remove unbound proteins. Soluble IgG or an anti-LILRB2 blocking antibody (Anti-RB2) was added at 20 μg/mL along with 50,000 LILRB2 chimeric receptor reporter cells (B2RC) and incubated for 24 hours. GFP⁺ cells were measured by flow cytometry.

FIG. 3: EphA7 activates LILRB2 chimeric receptor reporter cells. Proteins (SEMA4A as a positive control, and EphA7) were coated at 20 μg/mL for 18 hours at 37° C. Protein coated wells were washed with PBS three times to remove unbound proteins. Soluble IgG or an anti-LILRB2 blocking antibody (Anti-RB2) was added at 20 μg/mL along with 50,000 LILRB2 chimeric receptor reporter cells (B2RC) and incubated for 24 hours. GFP⁺ cells were measured by flow cytometry.

FIG. 4: Galectin-1 activates LILRB2 chimeric receptor reporter cells. Proteins (SEMA4A as a positive control, and Galectin-1 or Gal1) were coated at 20 μg/mL for 18 hours at 37° C. Protein coated wells were washed with PBS three times to remove unbound proteins. Soluble IgG or an anti-LILRB2 blocking antibody (Anti-RB2) was added at 20 μg/mL along with 50,000 LILRB2 chimeric receptor reporter cells (B2RC) and incubated for 24 hours. GFP⁺ cells were measured by flow cytometry.

FIGS. 5A-B: CLDN22 may interact with LILRB2. (FIG. 5A) Recombinant LILRB2-Fc protein binds to 293T cells that stably express hCLDN22 as detected by flow cytometry (B2Fc+). (FIG. 5B) Recombinant LILRB2-Fc protein was mixed with 293T cells (293T.WT), 293T cells that stably express hCLDN22 (293T.hC22), hCLDN22 with ECD1 deletion (293T.hC22ECD1 Del) or hCLDN22 with ECD2 deletion (293T.hC22ECD2Del). The binding of LILRB2-Fc may be dependent on both ECD1 and ECD2 of CLDN22.

FIGS. 6A-B: SCG2 activates and binds to LILRB4 chimeric receptor reporter cells. (FIG. 6A) Proteins (ApoE as a positive control, and SCG2) were coated at 20 μg/mL for 18 hours at 37° C. Protein coated wells were washed with PBS three times to remove unbound proteins. Soluble IgG (IgG-LALAPG) or an anti-LILRB4 blocking antibody (B4-LALAPG) was added at 20 μg/mL along with 50,000 LILRB4 chimeric receptor reporter cells (B4RC) and incubated for 24 hours. GFP⁺ cells were measured by flow cytometry. (FIG. 6B) Recombinant soluble SCG2-His protein (10 μg/mL) was added to LILRB4 chimeric receptor reporter cells for 90 minutes at 4° C. Cells were washed and stained with mouse Fc blocker and either Iso-APC or His-APC. SCG2-His⁺ cells were measured by flow cytometry.

FIGS. 7A-D: EphA4 activates LILRB5 chimeric receptor reporter cells. (FIG. 7A) Proteins (hGalectin-4 or hGal4 as a positive control, and EphA4) were coated at 20 μg/mL for 18 hours at 4° C. Protein coated wells were washed with PBS three times to remove unbound proteins. Soluble IgG or an anti-LILRB5 blocking antibody (Anti-RB5) was added at 20 μg/mL along with 50,000 LILRB5 chimeric receptor reporter cells (B5RC) and incubated for 24 hours. GFP⁺ cells were measured by flow cytometry. (FIG. 7B) LILRB5 blocking antibody (B5-27), human Ephrin A5 (hEphrinA5) or mouse Ephrin B2 (mEphrinB2) were added to the LILRB5 reporter cells (B5RC), which were then added to wells coated with hGalectin4, hEphA4 or mEphA4. GFP⁺ LILRB5 reporter cells were measured by flow cytometry. The result suggests that B5-27 is an effective antagonistic blocking antibody against LILRB5 because it blocks LILRB5 activation induced by a ligand (either Galectin4 or EphA4). It also suggests that the interaction between EphA4 (mouse or human) and LILRB5 is specific because anti-LILRB5 and the known EphA4 ligands (EphrinA5 and Ephrin B2) can block EphA4-induced LILRB5 activation; in contrast, EphrinA5 or Ephrin B2 cannot block Galectin4-induced LILRB5 activation. (FIG. 7C) Binding of LILRB5-hFc to mock or EphA4 transfected 293T cells were detected by flow cytometry. For left scatter plots of flow cytometry data, the x-axis GFP indicates the level of 293T cells transfected by mock or EphA4-encoding plasmid, and the y-axis indicates the binding of LILRB5-hFc to the transfected cells. For the right histogram plot, the y-axis of GFP^{hi}/GFP⁻ indicates the ratio of LILRB5 binding to GFp^{hi} cells versus to GFP-cells. (FIG. 7D) Coculture of 293T-EphA4 with LILRB5 reporter cells activates the LILRB5 reporter cells as determined by flow cytometry.

FIGS. 8A-C: EphA7 activates and binds to LILRB5 chimeric receptor reporter cells. (FIG. 8A) Proteins (hGalectin-4 or hGal4 as a positive control, and EphA7) were coated at 20 μg/mL for 18 hours at 4° C. Protein coated wells were washed with PBS three times to remove unbound proteins. Soluble IgG or an anti-LILRB5 blocking antibody (Anti-RB5) was added at 20 μg/mL along with 50,000 LILRB5 chimeric receptor reporter cells (B5RC) and incubated for 24 hours. GFP⁺ cells were measured by flow cytometry. (FIG. 8B) LILRB5 blocking antibody (B5-27), human Ephrin A5 (hEphrinA5) and mouse Ephrin B2 (mEphrinB2) were added to the LILRB5 reporter cell (B5RC) media before the cells in media with the above additions were added to wells coated with hGalectin4 or hEphA7. GFP⁺ LILRB5 reporter cells were measured by flow cytometry. (FIG. 8C) Binding of LILRB5-hFc to mock (293T-WT) or hEphA7 infected 293T cells (293T-hEphA7) were detected by flow cytometry.

FIG. 9: CST2 activates LILRB5 chimeric receptor reporter cells. Proteins (hGalectin-4 or hGal4 as a positive control, and CST2) were coated at 20 μg/mL for 18 hours at 37° C. Protein coated wells were washed with PBS three times to remove unbound proteins. Soluble IgG or an anti-LILRB5 blocking antibody (Anti-RB5) was added at 20 μg/mL along with 50,000 LILRB5 chimeric receptor reporter cells (B5RC) and incubated for 24 hours. GFP⁺ cells were measured by flow cytometry.

FIGS. 10A-D: CLDN22 activates LILRB5 chimeric receptor reporter cells. (FIG. 10A) Coated recombinant human CLDN22 protein (hCLDN22) activates LILRB5 reporter cells and the interaction is blocked by an LILRB5-LALAPG antibody (B5-LALPG) but not a control IgG (IgG-LALAPG). (FIG. 10B) Recombinant LILRB5-Fc protein binds to 293T-hCLDN22 cells and is detected by flow cytometry. (FIG. 10C) Recombinant LILRB5-Fc protein was mixed with 293T cells (293T.WT), 293T cells that stably express hCLDN22 (293T.hC22), hCLDN22 with ECD1 deletion (293T.hC22ECD1Del) or hCLDN22 with ECD2 deletion (293T.hC22ECD2Del). The binding is dependent on both ECD1 and ECD2 of CLDN22. (FIG. 10D) Coculture of 293T-hCLDN22 with LILRB5 reporter cells activates LILRB5 reporter cells by flow cytometry.

FIG. 11: Elastin activates LILRB5 chimeric receptor reporter cells. Proteins (hGalectin-4 or hGal4 as a positive control, and Elastin or ELN) were coated at 20 μg/mL for 18 hours at 37° C. Protein coated wells were washed with PBS three times to remove unbound proteins. Soluble IgG or an anti-LILRB5 blocking antibody (B5-27) was added at 20 μg/mL along with 50,000 LILRB5 chimeric receptor reporter cells (B5 RC) and incubated for 24 hours. GFP⁺ cells were measured by flow cytometry.

FIGS. 12A-D: Determination of interactions between LILRB family members and their potential protein ligands using ELISA. Ligand proteins (EphA7, EphA4, Galectin-4, and Galectin-7) were coated in wells in a high-binding 96-well plate (corning) at concentration of 2 μg/ml over night at 4° C. Coated plates were blocked using 2% BSA in PBS, pH7.4, for 1hour, then LILRB2 or LILRB5 (with Fc fusion) recombinant proteins were added in titrations of 3-fold down from 20 μg/ml for incubation at 25° C. for two hours. Binding signals were detected using anti-human Fc specific antibody conjugated with HRP (Jackson ImmunoResearch) using TMB substrate (ThermoFisher) by reading absorbance at 450 nm (Y-Axis). Binding affinity (EC₅₀, in the FIGS. 12C-D) was estimated using 4-parameter curve fitting with GraphPad Prism program.

FIGS. 13A-C: Interactions between LILRB5 and EphA2, EphA4, EphA7, EphB1 and EphB4 proteins using ELISA. Each of tested EphA or B proteins was coated in wells in a high-binding 96-well plate (corning) at concentration of 2 μg/ml over night at 4° C. Coated plates were blocked using 2% BSA in PBS, pH7.4, for 1 hour, then LILRB5 (with Fc or 6XHIS tag at C-terminus) recombinant extracellular domain (ECD) protein was added at 10 μg/ml (FIG. 13A) or in titrations of 3-fold down from 20 μg/ml for incubation at 25° C. for two hours. Binding signals were detected using anti-human Fc specific antibody conjugated with HRP (Jackson ImmunoResearch) or anti-His antibody using TMB substrate (ThermoFisher). A450 nm was read using a plate reader at 450 nm (Y-Axis). Binding affinity (EC₅₀, in the FIGS. 13B-C) was estimated using 4-parameter curve fitting with GraphPad Prism program.

FIG. 14: Galectin-1 activation of LILRA chimeric receptor reporter cells. Galectin-1 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 15: Galectin-2 activation of LILRA chimeric receptor reporter cells. Galectin-2 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 16: Galectin-3 activation of LILRA chimeric receptor reporter cells. Galectin-3 were coated at 20 g/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 17: Galectin-4 activation of LILRA chimeric receptor reporter cells. Galectin-4 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 18: Galectin-7 activation of LILRA chimeric receptor reporter cells. Galectin-7 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 19: Galectin-8 activation of LILRA chimeric receptor reporter cells. Galectin-8 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 20: Galectin-9's effect on LILRA chimeric receptor reporter cells. Galectin-9 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 21: Galectin-10 activation of LILRA chimeric receptor reporter cells. Galectin-10 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 22: Galectin-12 activation of LILRA chimeric receptor reporter cells. Galectin-12 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 23: Galectin-13 activation of LILRA chimeric receptor reporter cells. Galectin-13 were coated at 20 μg/mL for 18 hours at 37° C., and then washed with PBS three times to remove unbound proteins. 50,000 indicated LILRA chimeric receptor reporter cells were incubated for 24 hours. GFP⁺ cells were measured by flow cytometry.

FIG. 24: Galectins activation of LILRB1 and LILRB2 chimeric receptor reporter cells. Proteins (indicated anti-LILRB antibodies as positive controls, human CLDN1 as negative controls, and indicated Galectins) were coated at 20 μg/mL for 18 hours at 37° C. Protein wells were washed with PBS three times to remove unbound proteins. 50,000 LILRB1 or LILRB2 chimeric receptor reporter cells were incubated for 24 hours. GFP⁺ cells were measured by flow cytometry.

FIG. 25: Galectins activation of LILRB3 and LILRB4 chimeric receptor reporter cells. Proteins (indicated anti-LILRB antibodies as positive controls, human CLDN1 as negative controls, and indicated Galectins) were coated at 20 μg/mL for 18 hours at 37° C. Protein wells were washed with PBS three times to remove unbound proteins. 50,000 LILRB3 or LILRB4 chimeric receptor reporter cells were incubated for 24 hours. GFP⁺ cells were measured by flow cytometry.

FIG. 26: Galectins activation of LILRB5 and LAIR1 chimeric receptor reporter cells. Proteins (indicated anti-LILRB5 or anti-LAIR1 antibodies as positive controls, human CLDN1 as negative controls, and indicated Galectins) were coated at 20 μg/mL for 18 hours at 37° C. Protein wells were washed with PBS three times to remove unbound proteins. 50,000 LILRB5 or LAIR1 chimeric receptor reporter cells were incubated for 24 hours. GFP⁺ cells were measured by flow cytometry.

FIG. 27: Galectin-4's activation on LILRB chimeric receptor reporter cells can be blocked by lactose but not maltose, suggesting that the effect of Galectin-4 is mediated by its binding to β-galactoside sugars on LILRBs. When applied, galectin-4 was coated at 20 μg/mL for 18 hours at 37° C. and then washed with PBS three times to remove unbound proteins. The indicated concentrations of lactose or maltose were then added to the culture well, in which 50,000 LILRB chimeric receptor reporter cells were incubated for 24 hours. GFP⁺ cells were measured by flow cytometry.

FIG. 28: Galectin activated LILRB chimeric receptor reporter cells can be employed to identify antagonist anti-LILRB antibodies. Galectin-4 were coated at 20 μg/mL for 18 hours at 37° C. Protein wells were washed with PBS three times to remove unbound proteins. Soluble IgG or indicated different anti-LILRB4 antibodies were added at 20 μg/mL along with 50,000 LILRB4 chimeric receptor reporter cells and incubated for 24 hours. GFP⁺ cells were measured by flow cytometry.

FIG. 29. Summary of induction of LILRA and LILRB chimeric receptor reporter cells by galectins. Positive induction of indicated reporter cells by indicated galectins is demonstrated as green filled cells.

### DESCRIPTION OF ILLUSTRATIVE EMBODIMENTS

Targeted therapy may induce rapid tumor regression, whereas immunotherapy may achieve long-lasting anti-tumor effects. Thus, it would be ideal to identify molecular targets that enable the combination of the strengths of targeted therapy and immunotherapy. The function of leukocyte immunoglobulin-like receptor family (LILRs) that are expressed on both immune cells and cancer cells remain to be fully understood.

LILRs proteins are primate-specific, type I transmembrane glycoproteins. The extracellular Ig-like domains of these receptors bind ligands, and their intracellular immunoreceptor tyrosine-based activation motifs (ITAMs) or immunoreceptor tyrosine-based inhibitory motifs (ITIMs) can result in immune modulation. It has been suggested that the LILRB family is becoming the next wave of immune checkpoint targets for cancer treatment. Therefore, it is important to identify ligands for LILRBs and develop anti-LILRB blocking antibodies that can inhibit the ligand-induced LILRB activation, signaling, and function. We previously described the development of chimeric receptor reporter cells for several LILRBs and LAIR1¹⁻⁴.

Here, the inventors describe multiple proteins that can activate LILR receptors. These results are based on a screening system for LILR antagonist and blocking antibodies. Because individual LILR receptors may be able to be activated by more than one ligand or extracellular protein, some anti-LILR blocking antibodies may only block LILR activation induced by a certain but not all of these potential ligands/extracellular proteins. This system provides a new opportunity to screen LILRB antagonist and blocking antibodies, and to evaluate the blocking abilities of existing anti-LILR blocking antibodies in the presence of novel ligands/extracellular activators. These potential ligands/extracellular proteins of LILRs may also be targets for anti-cancer therapeutics development.

Therefore, embodiments of the present disclosure provide methods of identifying LILR antagonist (e.g., anti-LILR antibodies) specifically targeting ligand-induced LILR reporter activation. Provided are multiple proteins that can activate LILR receptors, based on which a screening system for LILR antagonists and blocking antibodies have been developed.

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. DEFINITIONS

As used herein, the singular forms “a”, “an” and “the” include plural references unless the context clearly dictates otherwise.

Throughout this application, the term “about” is used to indicate that a value includes the inherent variation of error for the device, the method being employed to determine the value, or the variation that exists among the study subjects.

Autoimmune disease includes, without limitation, rheumatoid arthritis, Crohn's disease, multiple sclerosis, autoimmune diabetes, systemic lupus erythematosus, lupus vulgaris, thyroiditis, Addison's Disease, hemolytic anemia, antiphospholipid syndrome, dermatitis, allergic encephalomyelitis, glomerulonephritis, Goodpasture's Syndrome, Graves' Disease, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Pulmonary Inflammation, Guillain-Barre Syndrome, and autoimmune inflammatory eye disease. Preferably, in the subject method, the subject is human. In one embodiment, the polypeptide is administered to the subject during a flare-up of an autoimmune attack. The method may further comprise administration of additional immunosuppressive drugs, e.g., cytotoxic agents, cyclosporine, methotrexate, azathioprine, and corticosteroids.

As used herein, “antagonist” or “inhibitor” of LILRB activation refers to any substance that can block or decrease the activation of LILRB in the presence of an LILRB ligand, e.g., ApoE. In certain embodiments, the antagonist or inhibitor can be protein, e.g., antibodies. In certain embodiments, the antagonist or inhibitor can be small molecule, e.g., a chemical compound. In certain embodiments, the antagonist or inhibitor decreases the activation of LILRB by at least 20%, 30%, 40%, 50%, 60%, 70%, 80% or 90% as compared to a reference level, e.g., the activation level of LILRB in the presence of LILRB ligand but in the absence of the antagonist or inhibitor.

The term “antibody” as used herein includes any immunoglobulin, monoclonal antibody, polyclonal antibody, multi-specific antibody, or bispecific (bivalent) antibody that binds to a specific antigen (or multiple antigens). A native intact antibody comprises two heavy chains and two light chains. Each heavy chain consists of a variable region (V_{H}) and a first, second, and third constant region (C_{H}1, C_{H}2, C_{H}3), while each light chain consists of a variable region (V_{L}) and a constant region (CL). Mammalian heavy chains are classified as α, δ, ε, γ, and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding and are often referred to as Fv (for variable fragment) or Fv fragment. The variable regions in both chains generally contains three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Chothia, Kabat, or Al-Lazikani (Chothia, C. et al., J Mol Biol 186 (3): 651-63 (1985); Chothia, C. and Lesk, A. M., J Mol Biol, 196:901 (1987); Chothia, C. et al., Nature 342 (6252): 877-83 (1989); Kabat E. A. et al., National Institutes of Health, Bethesda, Md. (1991); Al-Lazikani, B., Chothia, C., Lesk, A. M., J Mol Biol 273 (4): 927 (1997)). The three CDRs are interposed between flanking stretches known as framework regions (FRs), which are more highly conserved than the CDRs and form a scaffold to support the hypervariable loops. The constant regions of the heavy and light chains are not involved in antigen-binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain) in human, and IgG1 (γ1 heavy chain), IgG2a (γ2a heavy chain), IgG2b (γ2b heavy chain), and IgG3 (γ3 heavy chain) in mouse. As used herein, antibodies also include antigen-binding fragments, i.e., a portion of a protein which is capable of binding specifically to an antigen. In certain embodiment, the antigen-binding fragment is derived from an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a single domain antibody (sdAb), a camelid antibody or a nanobody, a domain antibody, and a bivalent domain antibody.

The term “cancer” refers to a condition or disorder in which cells grow and divide at unregulated, quickened pace. Examples of cancer include acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, cervical cancer, chronic lymphocytic leukemia, chronic myelogenous leukemia, colon cancer, emphysema, endometrial cancer, ependymoma, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukemia, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, vaginal cancer.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ, e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell; a cell from an endocrine system or organ, e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte); a cell from a nervous system or organ, e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph); a cell from a respiratory system or organ, e.g., a pneumocyte (a type I pneumocyte and a type II pneumocyte), a clara cell, a goblet cell, and an alveolar macrophage; a cell from circular system or organ (e.g., myocardiocyte and pericyte); a cell from digestive system or organ, e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, and a liver cell (e.g., a hepatocyte and Kupffer cell); a cell from integumentary system or organ, e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell), a muscle cell (e.g., myocyte), an adipocyte, a fibroblast, and a tendon cell; a cell from urinary system or organ (e.g., a podocyte, a juxtaglomerular cell, an intraglomerular mesangial cell, an extraglomerular mesangial cell, a kidney proximal tubule brush border cell, and a macula densa cell); and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The inflammatory disorder includes, without limitation, (i) inflammatory diseases such as chronic inflammatory pathologies (including chronic inflammatory pathologies such as, but not limited to, sarcoidosis, chronic inflammatory bowel disease, ulcerative colitis, and Crohn's pathology); (ii) vascular inflammatory pathologies such as, but not limited to, disseminated intravascular coagulation, atherosclerosis, Kawasaki's pathology and vasculitis syndromes (such as, but not limited to, polyarteritis nodosa, Wegener's granulomatosis, Henoch-Schonlein purpura, giant cell arthritis and microscopic vasculitis of the kidneys); (iii) chronic active hepatitis; (iv) Sjogren's syndrome; (v) spondyloarthropathies such as ankylosing spondylitis, psoriatic arthritis and spondylitis, enteropathic arthritis and spondylitis, reactive arthritis and arthritis associated with inflammatory bowel disease; and (vi) uveitis. Preferably, in the subject method, the subject is human. The method can also be combined with administration of additional anti-inflammatory agents. Anti-inflammatory agents include, but are not limited to, any known nonsteroidal anti-inflammatory agent such as, salicylic acid derivatives (aspirin), para-aminophenol derivatives (acetaminophen), indole and indene acetic acids (indomethacin), heteroaryl acetic acids (ketorolac), arylpropionic acids (ibuprofen), anthranilic acids (mefenamic acid), enolic acids (oxicams) and alkanones (nabumetone) and any known steroidal anti-inflammatory agent which include corticosteriods and biologically active synthetic analogs with respect to their relative glucocorticoid (metabolic) and mineralocorticoid (electrolyte-regulating) activities. Additionally, other drugs used in the therapy of inflammation include, but are not limited to, autocoid antagonists such as histamine, bradykinin receptor antagonists, leukotriene and prostaglandin receptor antagonists, and platelet activating factor receptor antagonists.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “subject” refers to a human or any non-human animal (e.g., mouse, rat, rabbit, dog, cat, cattle, swine, sheep, horse or primate). A human includes pre-and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

### II. LILRs

The leukocyte immunoglobulin-like receptors (LILR) are a family of receptors possessing extracellular immunoglobulin-like domains. They are also known as CD85, ILTs and LIR, and can exert immunomodulatory effects on a wide range of immune cells. The human genes encoding these receptors are found in a gene cluster at chromosomal region 19q13.4. They include LILRA1, LILRA2, LILRA3, LILRA4, LILRA5, LILRA6, LILRB1, LILRB2, LILRB3, LILRB4, and LILRB5. A subset of LILRs recognize MHC class I molecules (also known as HLA class I in humans). Of these, the inhibitory receptors LILRB1 and LILRB2 show a broad specificity for classical and non-classical MHC alleles with preferential binding to β2m-associated complexes, particularly in the case of LILRB1. In contrast, the activating receptors LILRA1 and LILRA3 prefer b2m-independent free heavy chains of MHC class I, and in particular HLA-C alleles. LAIR1 (Leukocyte Associated Immunoglobulin Like Receptor 1, LAIR-1, CD305) is a member of both the immunoglobulin superfamily and the leukocyte-associated inhibitory receptor family with close relationship to LILRBs. The LAIR1 gene also maps to a region of 19q13.4 called the leukocyte receptor cluster, which contains at least 29 genes encoding leukocyte-expressed receptors of the immunoglobulin superfamily.

### A. LILRA1

Leukocyte immunoglobulin-like receptor, subfamily A (with TM domain), member 1 is a protein that in humans is encoded by the LILRA1 gene. It is found in a gene cluster at chromosomal region 19q13.4. The encoded protein is predominantly expressed in B cells, interacts with major histocompatibility complex class I ligands, and contributes to the regulation of immune responses. Alternative splicing results in multiple transcript variants encoding different isoforms.

### B. LILRA3

Leukocyte immunoglobulin-like receptor subfamily A member 3 (LILRA3) also known as CD85 antigen-like family member E (CD85e), immunoglobulin-like transcript 6 (ILT-6), and leukocyte immunoglobulin-like receptor 4 (LIR-4) is a protein that in humans is encoded by the LILRA3 gene located within the leukocyte receptor complex on chromosome 19q13.4. Unlike many of its family, LILRA3 lacks a transmembrane domain. The function of LILRA3 is currently unknown; however, it is highly homologous to other LILR genes, and can bind human leukocyte antigen (HLA) class I. Therefore, if secreted, the LILRA3 might impair interactions of membrane bound LILRs (such as LILRB1, an inhibitory receptor expressed on effector and memory CD8 T cells) with their HLA ligands, thus modulating immune reactions and influencing susceptibility to disease. Like the closely related LILRA1, LILRA3 binds to both normal and ‘unfolded’ free heavy chains of HLA class I, with a preference for free heavy chains of HLA-C alleles.

### C. LILRA6

Leukocyte immunoglobulin-like receptor subfamily A member 6 (LILRA6) also known as CD85 antigen-like family member E (CD85b) and immunoglobulin-like transcript 8 (ILT-8). It is predicted to enable inhibitory MHC class I receptor activity, to be involved in cytokine-mediated signaling pathway, and to be integral component of membrane. Predicted to be active in plasma membrane.

### D. LILRB1

Leukocyte immunoglobulin-like receptor subfamily B member 1 is a protein that in humans is encoded by the LILRB1 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. It is thought to control inflammatory responses and cytotoxicity to help focus the immune response and limit autoreactivity. Multiple transcript variants encoding different isoforms have been found for this gene.

### E. LILRB2

Leukocyte immunoglobulin-like receptor subfamily B member 2 is a protein that in humans is encoded by the LILRB2 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on myeloid cells where it binds to soluble or membrane-bound MHC class I molecules and transduces a negative signal that inhibits stimulation of an immune response through recruiting phosphatases. It is thought to control inflammatory responses and cytotoxicity to help focus the immune response and limit autoreactivity. Multiple transcript variants encoding different isoforms have been found for this gene.

### F. LILRB3

Leukocyte immunoglobulin-like receptor subfamily B member 3 is a protein that in humans is encoded by the LILRB3 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells and is believed to be a myeloid checkpoint. It is thought to control inflammatory responses and cytotoxicity to help focus the immune response and limit autoreactivity. Multiple transcript variants encoding different isoforms have been found for this gene.

### G. LILRB4

Leukocyte immunoglobulin-like receptor subfamily B member 4 is a protein that in humans is encoded by the LILRB4 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on monocytic myeloid cells where it binds to ApoE and fibronectin and transduces a negative signal that inhibits stimulation of an immune response through recruiting phosphatases. The receptor can also function in antigen capture and presentation. It is thought to control inflammatory responses and cytotoxicity to help focus the immune response and limit autoreactivity. Multiple transcript variants encoding different isoforms have been found for this gene.

### H. LILRB5

Leukocyte immunoglobulin-like receptor subfamily B member 5 (LILRB5) is a protein that in humans is encoded by the LILRB5 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). It was reported that LILRB5 binds to HLA-B7 and HLA-B27 heavy chains.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Results

As discussed above, the leukocyte Ig-like receptor subfamily (LILR) proteins are primate-specific, type I transmembrane glycoproteins. The extracellular Ig-like domains of these receptors bind ligands, and their intracellular immunoreceptor tyrosine-based activation motifs (ITAMs) or immunoreceptor tyrosine-based inhibitory motifs (ITIMs) can result in immune modulation. It was suggested that the LILRB family is becoming the next wave of immune checkpoint targets for cancer treatment. Therefore, it is important to identify ligands for LILRBs and develop anti-LILRB blocking antibodies that can inhibit the ligand-induced LILRB activation, signaling, and function. The inventors previously described the development of assays to identify ligands for LILRB. See WO2018/022881, which is herein incorporated in its entirety).

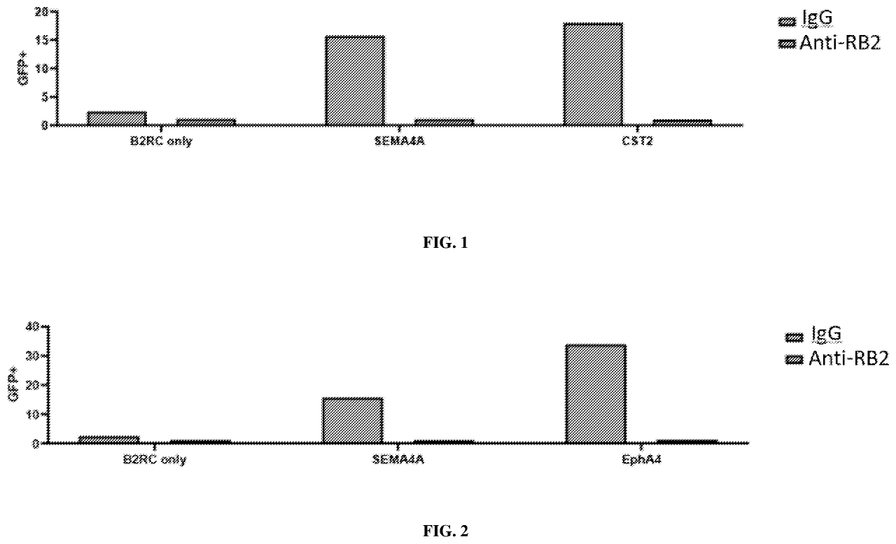

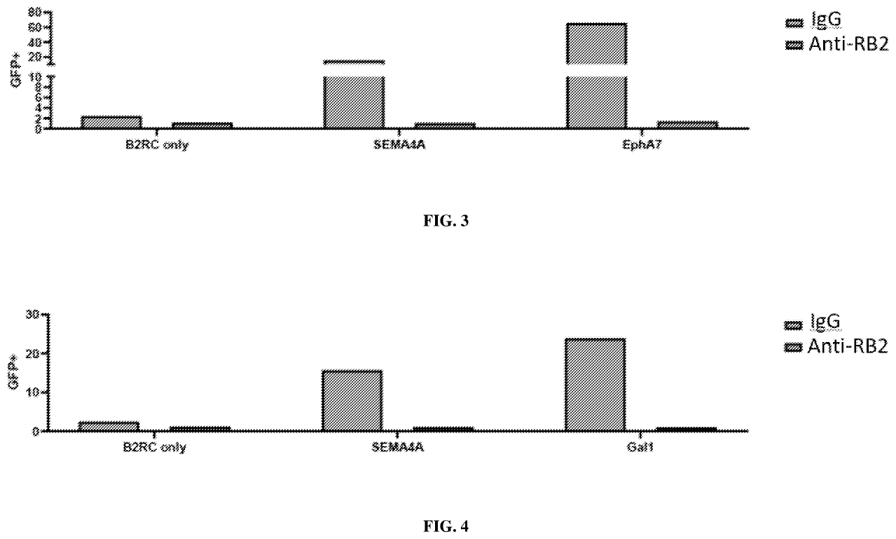

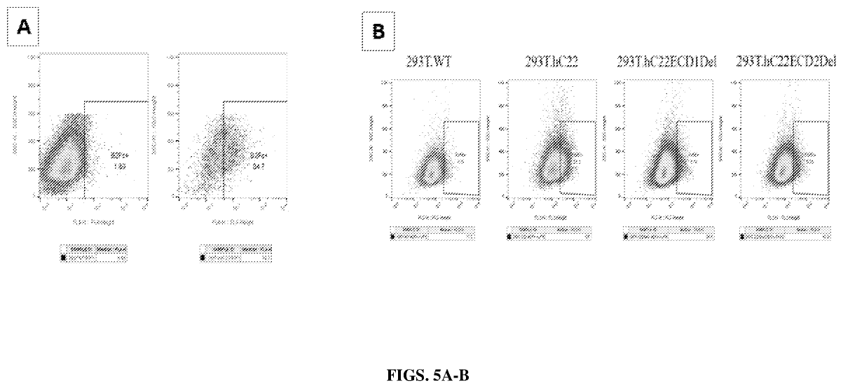

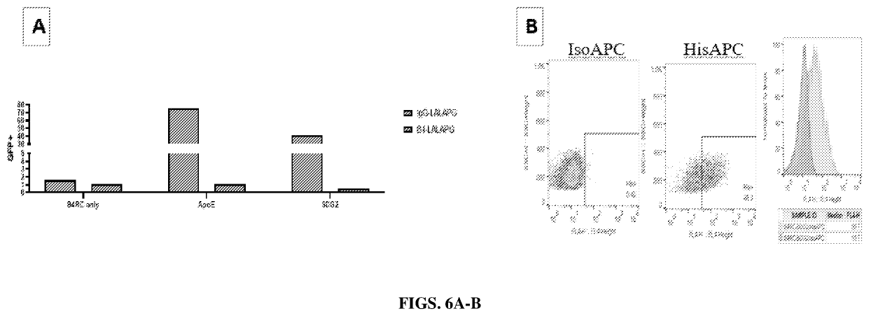

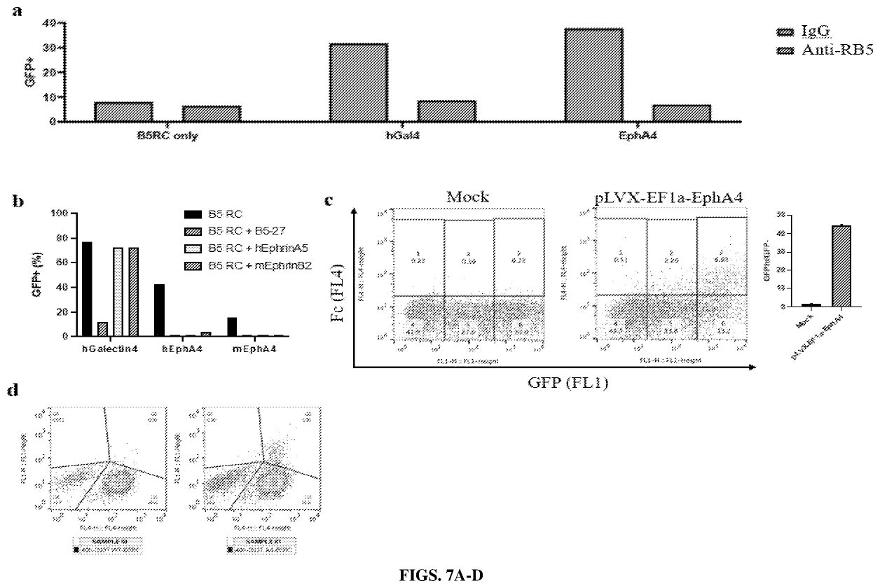

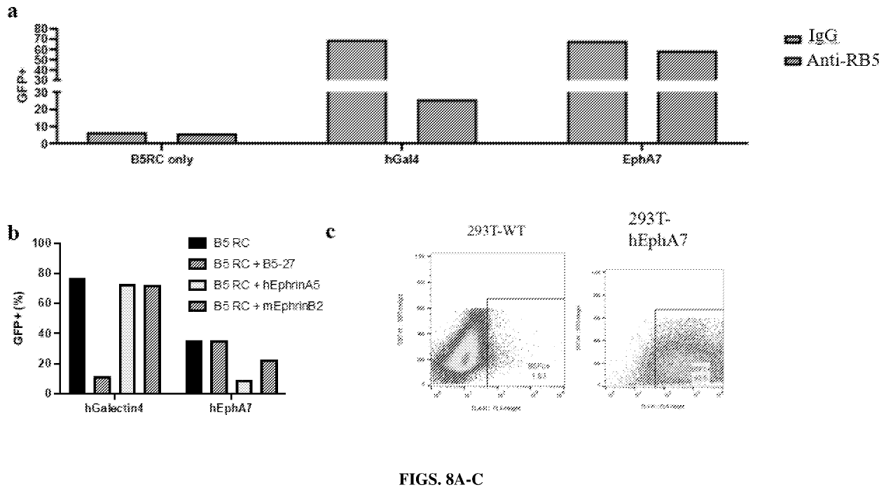

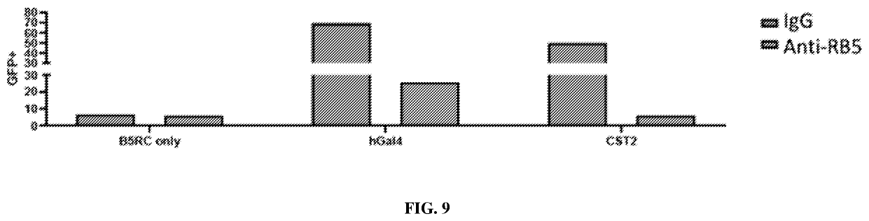

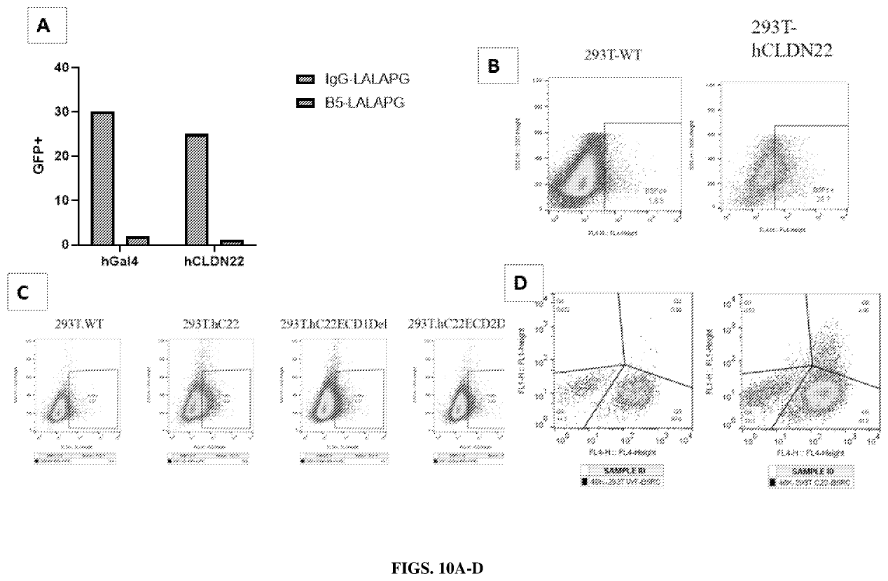

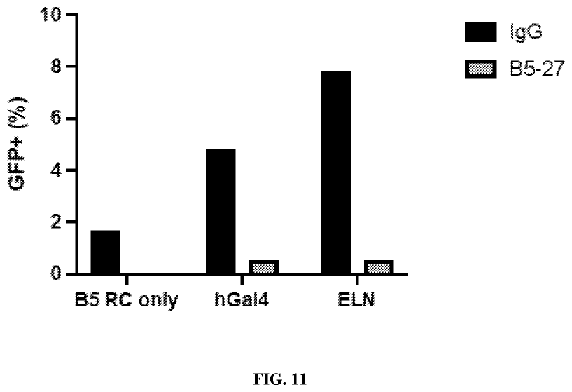

FIGS. 1-5 show that Cystatin SA (CST2), EPH Receptor A4 (EphA4), EPH Receptor A7 (EphA7), Galectin-1, and claudin 22 (CLDN22) can activate or bind LILRB2. LILRB2 chimeric receptor reporter cells in the presence of any of these proteins may be used to screen antagonists or blocking antibodies against LILRB2. FIG. 6 shows that SCG2 can bind and activate LILRB4. LILRB4 chimeric receptor reporter cells in the presence of SCG2 may be used to screen antagonists or blocking antibodies against LILRB4. FIGS. 7-11 show that EphA4, EphA7, CST2, CLDN22, and elastin can activate LILRB5. LILRB5 chimeric receptor reporter cells in the presence of any of these proteins may be used to screen antagonists or blocking antibodies against LILRB5.

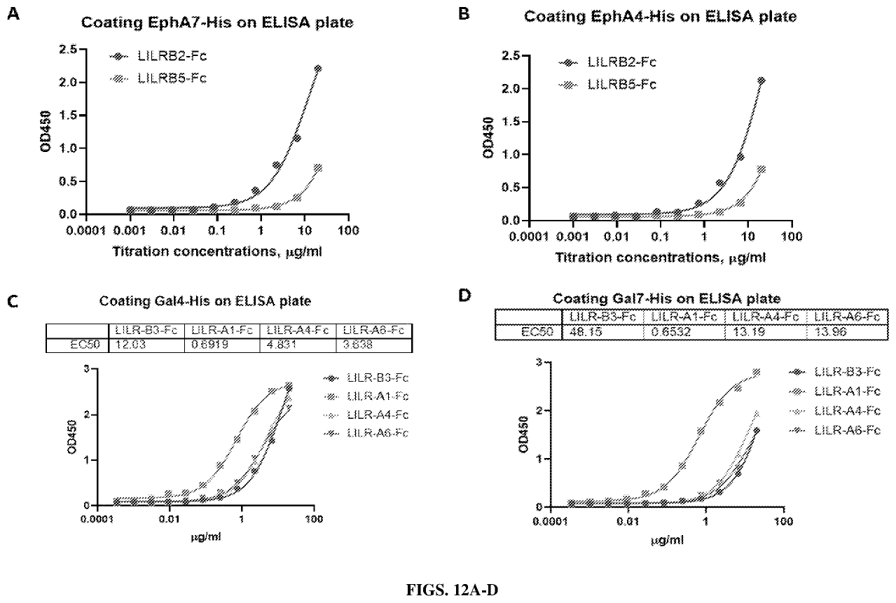

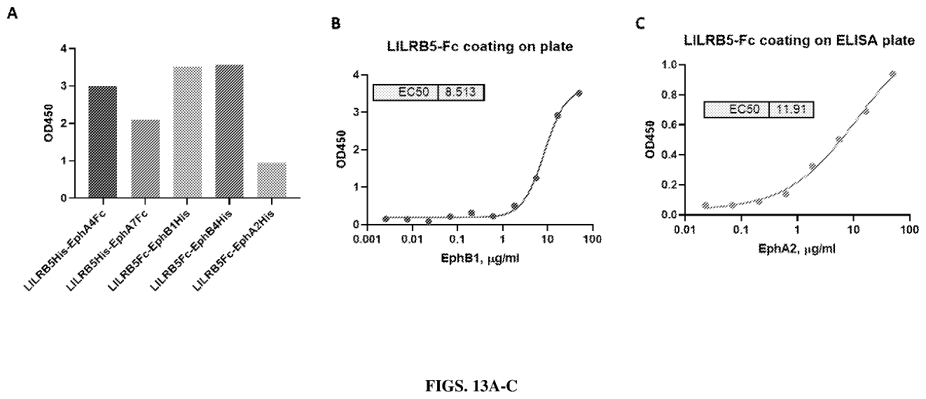

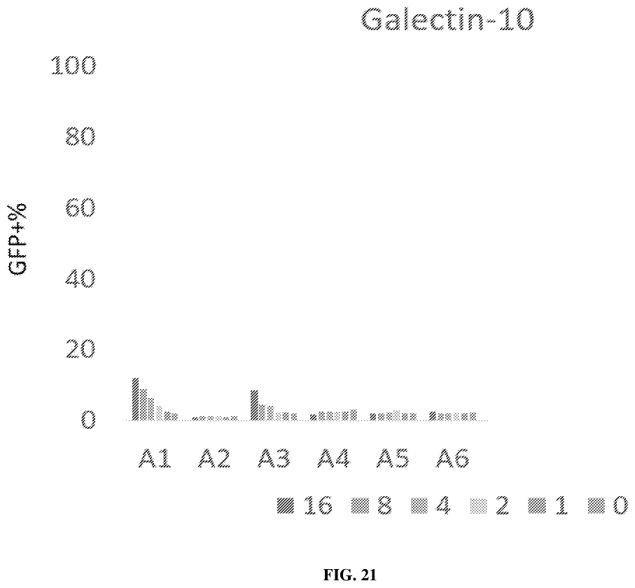

FIGS. 12A-D show that binding of LILRB family members and their potential protein ligands as determined using ELISA. Plates were coated with His labelled EphA7 (FIG. 21A); EphA4 (FIG. 12B); Gal4 (FIG. 12C); and Gal7 (FIG. 12D). FIGS. 13A-C show the binding between LILRB5 and EphA2, EphA4, EphA7, EphB1 and EphB4 proteins using ELISA.

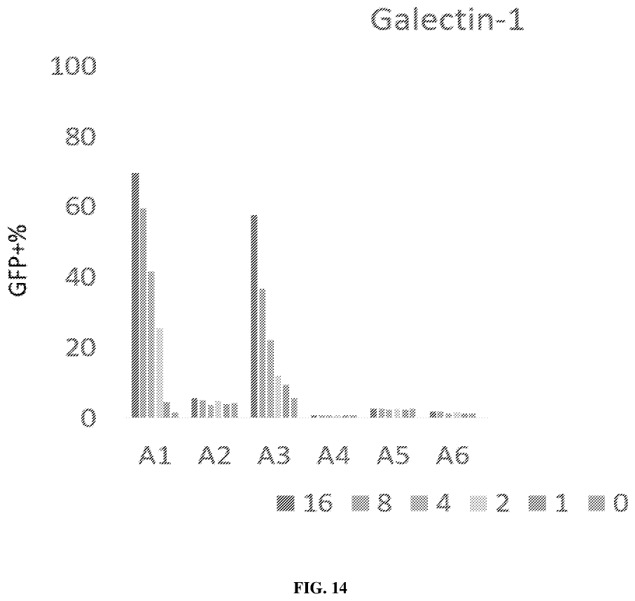

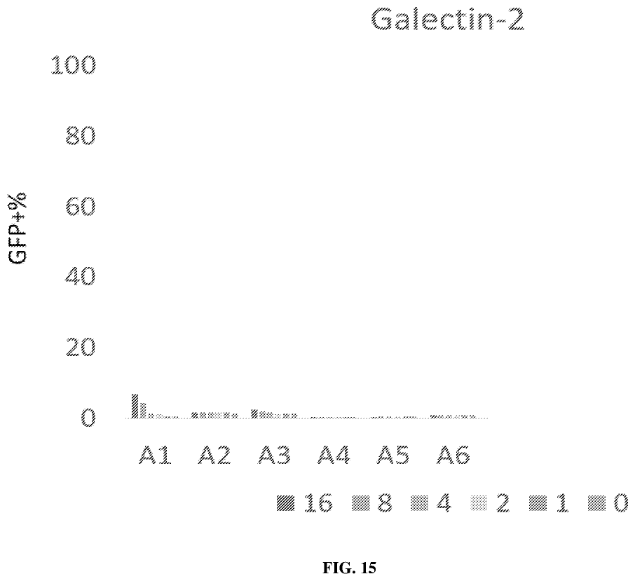

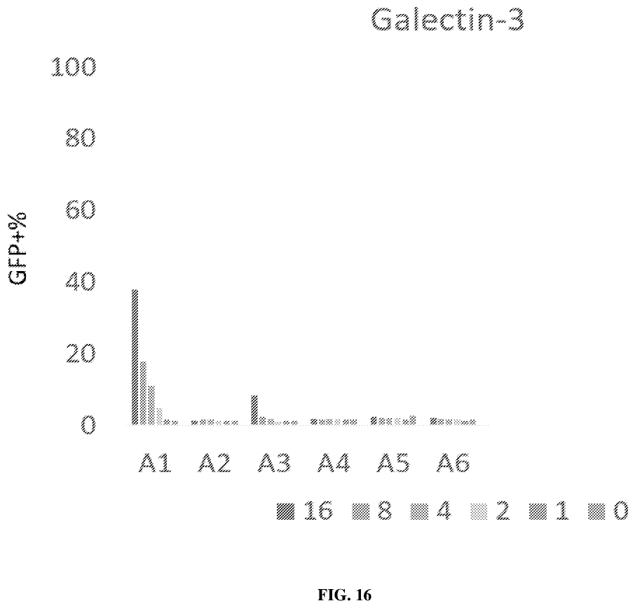

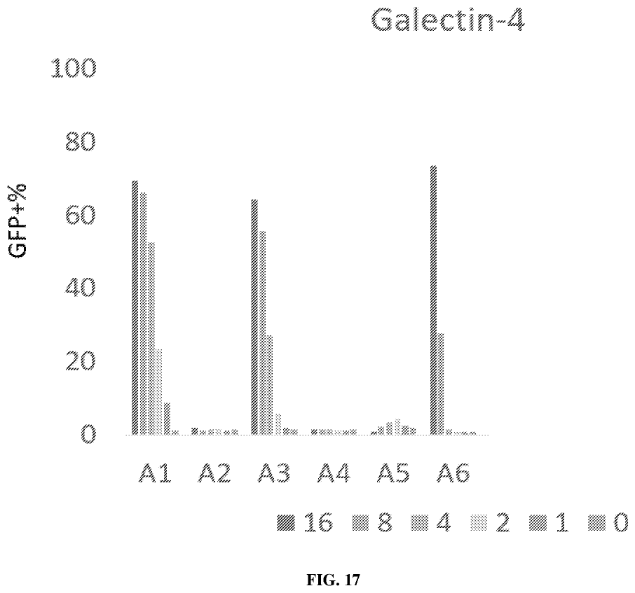

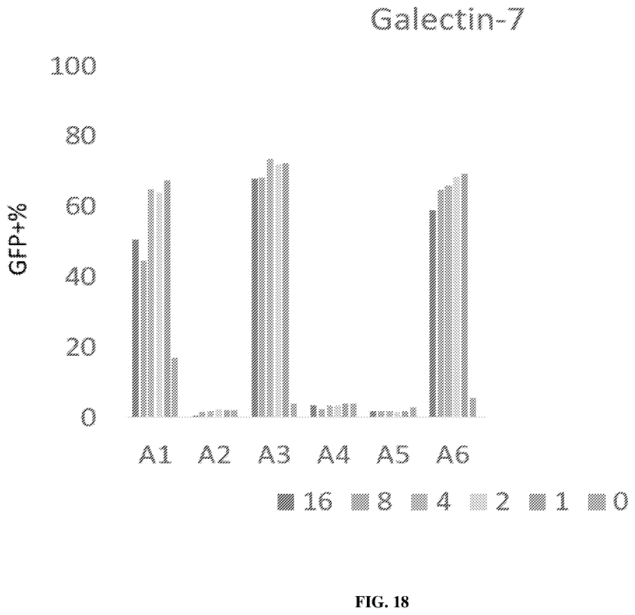

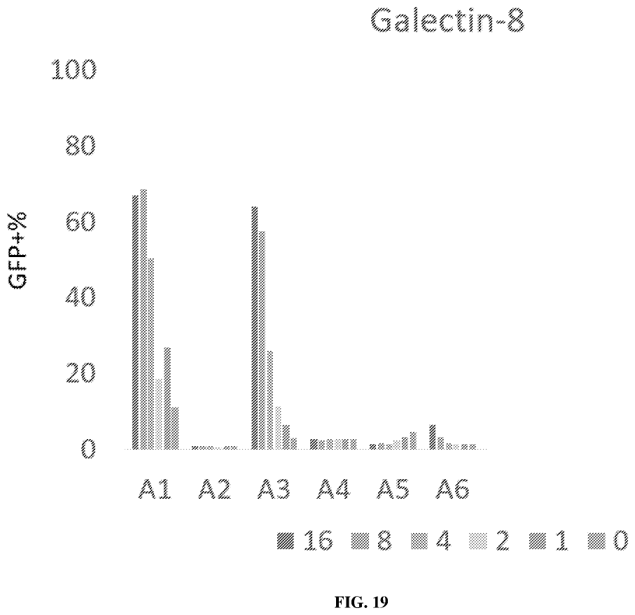

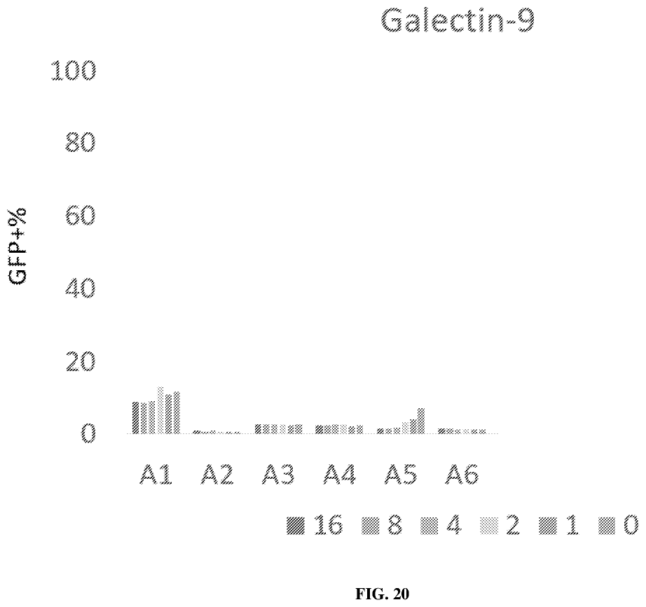

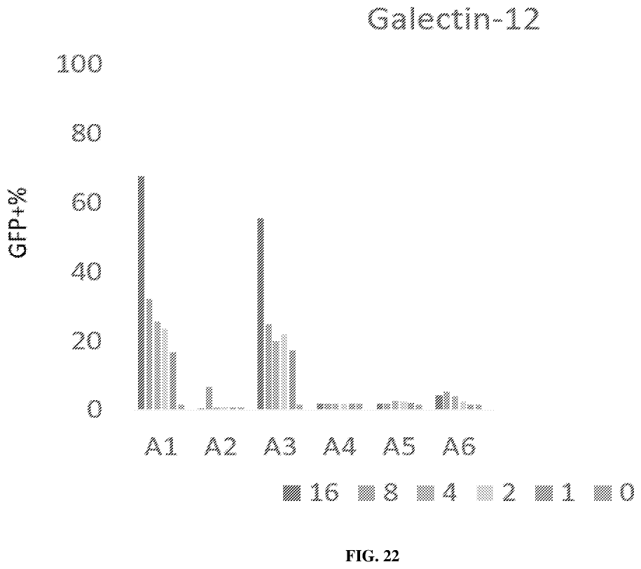

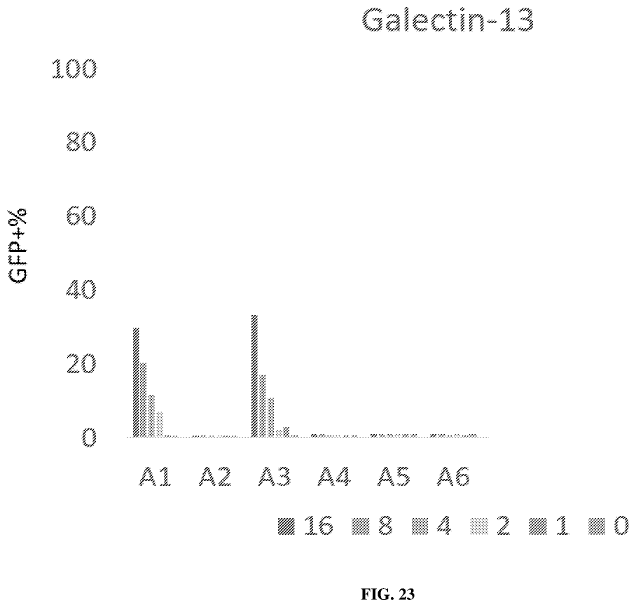

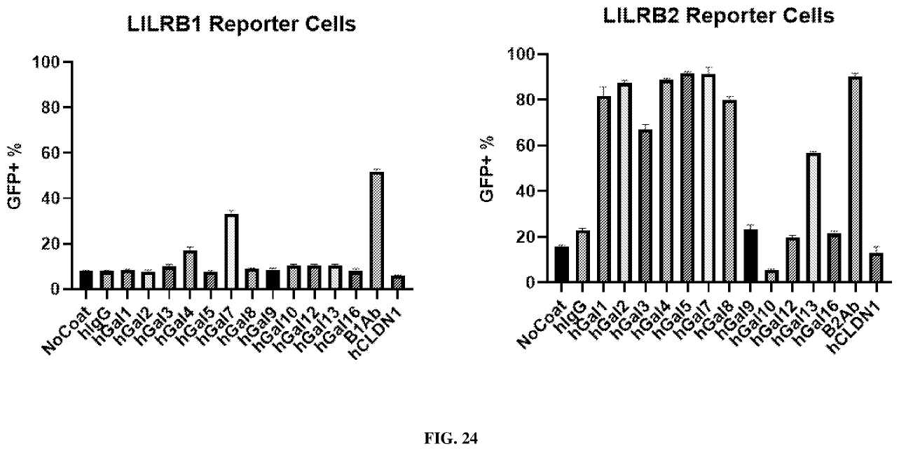

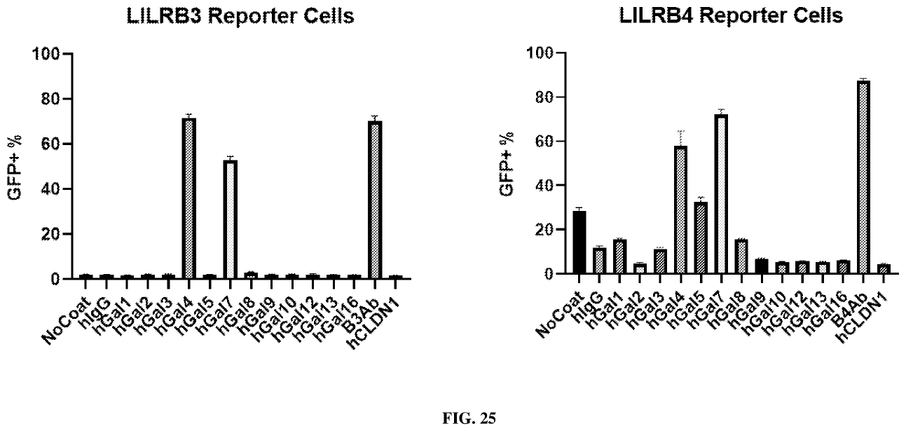

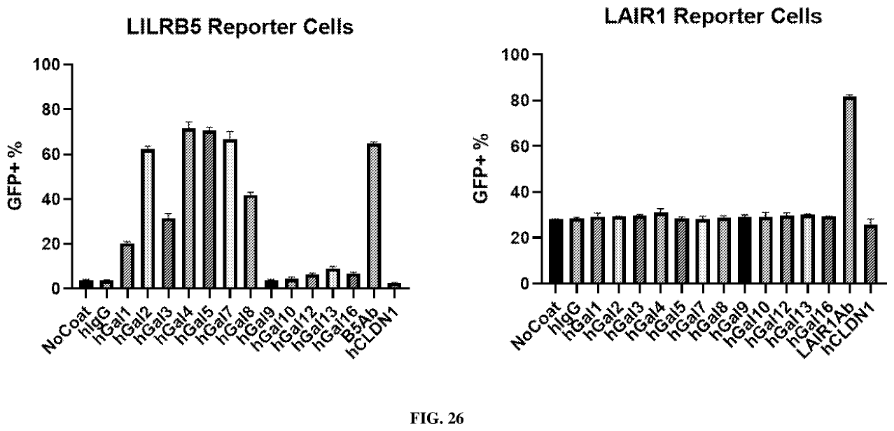

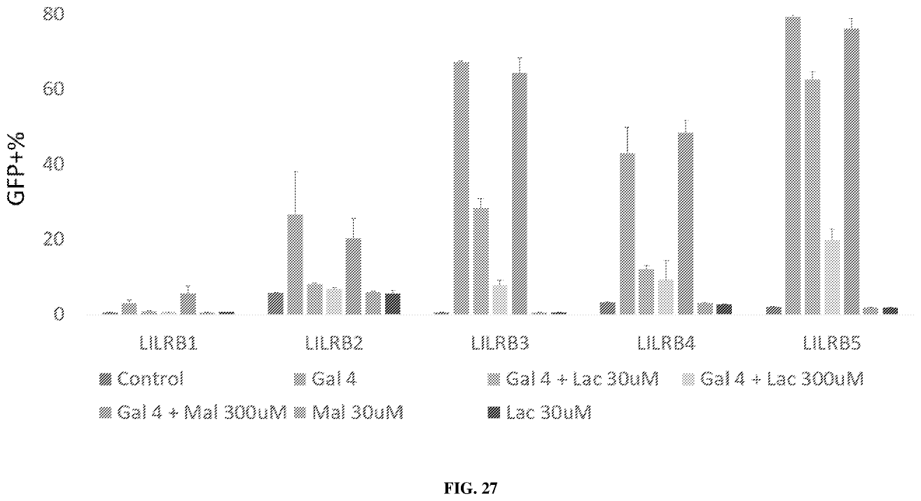

FIGS. 14-23 illustrate that that Galectin-1, galectin-3, galectin-4, galectin-7, galectin-8, galectin-10, galectin-12, or galectin-13 induced activation of LILRA1 chimeric receptor reporter cells as determined by flow cytometry. FIG. 24 (left) shows that Galectin-4 or galectin-7 induced activation of the LILRB1 chimeric receptor reporter cells. FIG. 24 (right) shows Galectin-1, galectin-2, galectin-3, galectin-4, galectin-5, galectin-7, galectin-8, galectin-12, or galectin-13 induced activation of the LILRB2 chimeric receptor reporter cells. FIG. 25 (left) shows Galectin-4 or galectin-7 induced activation of the LILRB3 chimeric receptor reporter cells. FIG. 25 (right) shows Galectin-4, galectin-5, or galectin-7 induced activation of the LILRB4 chimeric receptor reporter cells. FIG. 26 (left) shows Galectin-1, galectin-2, galectin-3, galectin-4, galectin-5, galectin-7, galectin-8, or galectin-12 induced activation of the LILRB5 chimeric receptor reporter cells. FIG. 26 (right) shows that only the control LAIR1 antibody activated LAIR1 chimeric receptor reporter cells.

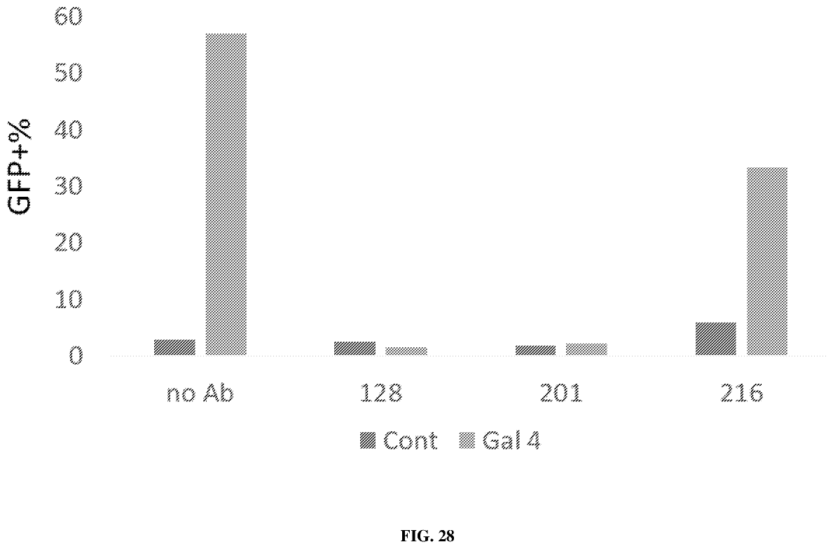

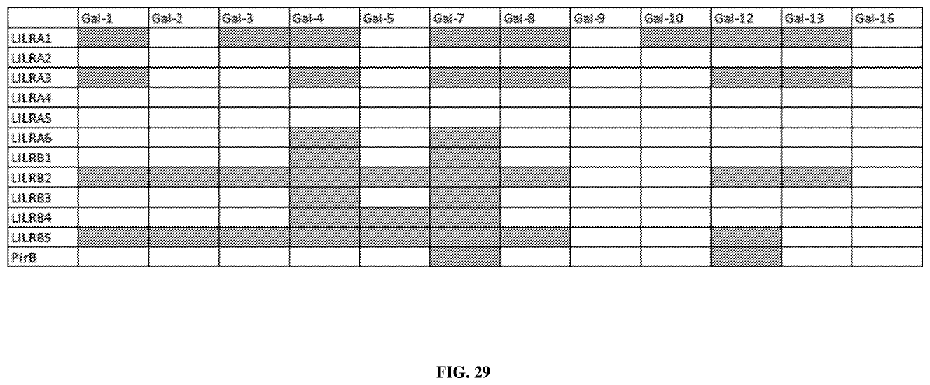

FIG. 27 shows that Galectin-4's activation on LILRB1-5 chimeric receptor reporter cells could be blocked by lactose, but not maltose, suggesting that the effect of Galectin-4 is mediated by its binding to β-galactoside sugars on LILRB1-5. FIG. 28 shows galectin activated LILRB chimeric receptor reporter cells can be used to identify antagonist anti-LILRB antibodies as illustrated by using LILRB4 reporter cells. FIG. 29 presents a summary table of the ability of tested galectins to induce certain LILRA, LILRB and PirB chimeric receptor reporter cells in the described assay. Positive induction of indicated reporter cells by indicated galectins is demonstrated as green filled cells.

### Example 2

### Materials and Methods

Chimeric receptor reporter assay. The inventors constructed a stable chimeric receptor reporter cell system to test the ability of a ligand to bind to the ECD of individual LILRBs and to trigger the activation or inhibition of the chimerically fused intracellular domain of paired immunoglobulin-like receptor B, which signals through the adaptor DAP-12 to activate the NFAT promoter. If an agonist or antagonist binds to the ECD and activates or suppresses the chimeric signaling domain, an increase or decrease, respectively, in GFP expression is observed. A competition assay as shown in figures was used to attest LILRB ligand blocking antibodies.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### IV. REFERENCES

The following references, to the extent that they provide exemplary procedural or other details supplementary to those set forth herein, are specifically incorporated herein by reference.
1. Deng M, Lu Z, Zheng J, et al. A motif in LILRB2 critical for Angptl2 binding and activation. Blood. 2014;124 (6): 924-935.
2. Kang X, Lu Z, Cui C, et al. The ITIM-containing receptor LAIR1 is essential for acute myeloid leukaemia development. Nat Cell Biol. 2015;17 (5): 665-677.
3. Deng M, Gui X, Kim J, et al. LILRB4 signalling in leukaemia cells mediates T cell suppression and tumour infiltration. Nature. 2018;562 (7728): 605-609.
4. Wu G, Xu Y, Schultz RD, et al. LILRB3 supports acute myeloid leukemia development and regulates T-cell antitumor immune responses through the TRAF2-cFLIP-NF-κB signaling axis. Nature Cancer. 2021.
5. Zhang Z, Hatano H, Shaw J, et al. The Leukocyte Immunoglobulin-Like Receptor Family Member LILRB5 Binds to HLA-Class I Heavy Chains. PLOS One. 2015;10 (6): e0129063.

What is claimed is:

1. A method of identifying a modulator of LILR activation comprising:
(a) contacting a reporter cell with a ligand of LILR and a candidate substance with potential to modulate LILR reporter activation; and
(b) detecting a level of LILR reporter activation in the reporter cell, wherein a change in the level of LILR reporter activation as compared to a reference level indicates that the candidate substance is a modulator of LILR activation.

2. The method of claim 1, wherein the reporter cell expresses a receptor comprising an extracellular domain of LILR.

3. The method of claim 1, wherein the cell is a mouse T-cell hybridoma cell.

4. The method of claim 2, wherein the LILR is further defined as LILRA1, LILRA3, LILRB1, LILRB2, LILR3, LILRB4, LILRB5, or LILRA6.

5. The method of claim 4, wherein (a) the ligand of LILRA1 is Galectin-1, Galectin-3, Galectin-4, Galectin-7, Galectin-8, Galectin-10, Galectin-12, or Galectin-13, (b) the ligand of LILRA3 is Galectin-1, Galectin-4, Galectin-7, Galectin-8, Galectin-12, or Galectin-13, or (c) the ligand of LILRA6 is Galectin-4 or Galectin-7.

6. The method of claim 4, wherein (a) the ligand of LILRB1 is Galectin-4 or Galectin-7, (b) the ligand of LILRB2 is Cystatin SA (CST2), EPH Receptor A4 (EphA4), EPH Receptor A7 (EphA7), Galectin-1, Galectin-2, Galectin-3, Galectin-4, Galectin-5, Galectin-7, Galectin-8, Galectin-12, Galectin-13 or claudin 22 (CLDN22), (c) the ligand of LILRB3 is Galectin-4 or Galectin-7, (d) the ligand of LILRB4 is SCG2, Galectin-4, Galectin-5 or Galectin-7, or (e) the ligand of LILRB5 is EphA4, EphA7, CST2, CLDN22, Galectin-1, Galectin-2, Galectin-3, Galectin-4, Galectin-5, Galectin-7, Galectin-8, or Galectin-12 or elastin.

7. The method of claim 2, wherein the receptor further comprises an intracellular domain of paired immunoglobulin-like receptor β (PILRβ).

8. The method of claim 7, wherein the ligand of PILRβ is Galectin-7 or Galectin-12.

9. The method of claim 2, wherein the receptor is expressed in the cell through a viral expression vector.

10. The method of claim 9, wherein the viral expression vector is a retroviral expression vector.

11. The method of claim 1, wherein the level of LILR activation is detected based on the morphology or mobility of the cell.

12. The method of claim 1, wherein the reporter cell expresses a reporter gene that encodes a protein that emits or generates a detectable label and is operably linked to a promoter regulated by activation of the receptor.

13. The method of claim 12, wherein the promoter is a nuclear factor of activated T cells (NFAT) promoter.

14. The method of claim 12, wherein the promoter is a CCL2 promoter, a CCL4 promoter, a CCL5 promoter, an IL-6R promoter, an IL-8 promoter, a gpl30 promoter, an OSM promoter, a TIMP-1/2 promoter, a TNF-R1/II promoter, a uPAR promoter or an arginase-1 promoter.

15. The method of claim 12, wherein the detectable label is a colorometric label, fluorescent label, bioluminescent label, or chemiluminescent label.

16. The method of claim 12, wherein the protein encoded by the reporter gene is GFP, YFP, RFP, or Luciferase.

17. The method of claim 12, wherein the detectable label is D-luciferin.

18. The method of claim 12, wherein detecting step comprises flow cytometry analysis or quantification of luminescence.

19. The method of claim 1, wherein the candidate substance is an antibody.

20. The method of claim 19, wherein the antibody is a monoclonal antibody, a chimeric antibody, a CDR-grafted antibody, a humanized antibody, a Fab, a Fab′, a F(ab′)2, a Fv, or a scFv.

21. The method of claim 19, wherein the antibody is a monoclonal antibody.

22. The method of claim 1, wherein the reference level is obtained in the reporter cell when it is contacted with the ligand of LILRB in the absence of the candidate substance.

23. The method of claim 1, wherein an increase in the level of LILRB activation as compared to the reference level indicates that the modulator is an agonist.

24. The method of claim 1, wherein a decrease in the level of LILRB activation as compared to the reference level indicates that the modulator is an antagonist.

25. The method of claim 1, wherein the candidate substance is linked to a substrate or a cell expressing FcR.

26. A composition comprising: a candidate LILR modulator;
a ligand of LILR; and
a reporter cell that expresses a receptor comprising an extracellular domain of LILR, wherein the reporter cell has a phenotype indicating LILR activation.

27. The composition of claim 26, wherein the candidate LILR modulator is an antibody.

28. The composition of claim 26, wherein the receptor further comprises an intracellular domain of PILR.

29. The composition of claim 26, wherein the reporter cell further comprises a reporter gene that encodes a protein that emits or generates a detectable label and that is operably linked to a promoter regulated by activation of the receptor, such as wherein the protein encoded by the reporter gene is GFP.

30. The composition of claim 26, further comprising a cell expressing FcR.

31. A composition comprising:
a candidate LILR modulator;
a cell expressing FcR; and
a reporter cell that expresses a receptor comprising an extracellular domain of LILR, wherein the reporter cell has a phenotype indicating LILR activation.

32. The composition of claim 31, wherein the candidate LILR modulator is an antibody.

33. The composition of claim 31, wherein the receptor further comprises an intracellular domain of PILR.

34. The composition of claim 31, wherein the reporter cell further comprises a reporter gene that encodes a protein that emits or generates a detectable label and that is operably linked to a promoter regulated by activation of the receptor.

35. The composition of claim 34, wherein the protein encoded by the reporter gene is GFP.

36. A method of treating cancer in a subject comprising administering an effective amount of an inhibitor of ligand-induced LILR activation to a subject.

37. The method of claim 36, wherein the inhibitor of ligand-induced LILR activation is an antibody.

38. The method of claim 36, wherein the inhibitor of ligand-induced LILR activation is a protein or peptide.

39. A method of treating autoimmune disease or inhibiting the onset of transplant rejection or treating an inflammatory disorder in a subject comprising administering an effective amount of an agonist of ligand-induced LILR activation to a subject.

40. The method of claim 39, wherein the agonist of ligand-induced LILR activation is an antibody.

41. The method of claim 39, wherein the agonist of ligand-induced LILR activation is a protein or peptide.
